(19) United States
(12) Patent Application Publication
(10) Pub. No.: US 2019/0159701 A1
(43) Pub. Date: May 30, 2019

(54) SYSTEM AND METHOD FOR USE IN TISSUE MONITORING AND ANALYSIS

(71) Applicant: CONTINUSE BIOMETRICS LTD., Tel Aviv (IL)

(72) Inventors: Yevgeny BEIDERMAN, Tel Aviv (IL); Zeev ZALEVSKY, Rosh HaAyin (IL); Sagi POLANI, Tel-Aviv (IL); Mark GOLBERG, Rehovot (IL); Javier GARCIA, Valencia (ES); Joaquin RUIZ-RIVAS ONSES, Madrid (ES); Martin SANZ SABATER, Valencia (ES)

(21) Appl. No.: 16/098,206

(22) PCT Filed: May 4, 2017
(86) PCT No.: PCT/IL2017/050493

§ 371 Date: Nov. 1, 2018

Related U.S. Application Data
(60) Provisional application No. 62/349,745, filed on Jun. 14, 2016.
(60) Provisional application No. 62/332,005, filed on May 5, 2016.

(51) Int. Cl. A61B 5/11; A61B 5/113; A61B 5/00; G06T 7/00; G01H 9/00

(52) U.S. Cl. CPC A61B 5/1128; G06T 2207/30048; A61B 5/1102; A61B 5/113; A61B 5/4806; A61B 5/4866; A61B 5/0077; A61B 5/7246; A61B 5/7455; A61B 5/0066; A61B 5/4509; A61B 5/0051; A61B 5/7278; G06T 7/0012; G01H 9/00; A61B 2576/00; G06T 2207/30041; A61B 5/1103

(57) ABSTRACT

A system and technique are presented, for use in remote sensing of acoustic and/or mechanical vibration data from an inspection region. The system comprises: input port for receiving input data comprising a sequence of image data pieces indicative of speckle patterns detected in light returning from an inspection region, at least one processing utility configured for processing and analyzing the input data, the at least one processing utility comprises: a correlation module configured for processing the sequence of image data piece and determining correlation function indicative of spatial correlations between speckle patterns in consecutive image data pieces, said correlation function being indicative of variation in position of points within an inspected region; a filtering module configured and operable for receiving and processing data on the determined correlation function for extracting data indicative on vibration having selected frequency range for determining data indicative of at least one of acoustic signals, movement and tactile vibrations of said inspection region.

### TECHNOLOGICAL FIELD

The invention is in the field of optical inspection and is of particular relevance to detection of signals associated mechanical and acoustic vibrations of biological tissue.

### BACKGROUND

Vibrations of various frequency ranges and amplitudes provide corresponding data. Accordingly, at frequency range of 20 Hz to 20 KHz vibrations may generate or be associated with acoustic signals; while in various frequency ranges and in accordance with amplitude thereof such vibrations may be sensed as mechanical vibrations of the sample. Both acoustic and mechanical vibrations of a sample may provide data on the sample.

For example, acoustic signals are commonly used in medical examination of patients for various conditions. Additionally, mechanical vibrations provide tactile data informing physicians about differences between tissue regions and allowing touch sensing of tissue parameters/condition. The detection of mechanical and/or acoustic signals generally provides a physician with non-invasive indication on biomechanical activity within the human body (or any other organism).

The commonly used stethoscope has become a symbol of a physician and provides detection of acoustic signals in a simple and non-invasive fashion. Additional techniques are being developed, providing electronic remote detection acoustic signals.

Several techniques have been developed or co-developed by the inventors of the present invention, providing remote sensing of movement. For example:

U.S. Pat. No. 8,638,991 presents a method for imaging an object. The method comprises imaging a coherent speckle pattern propagating from an object, using an imaging system being focused on a plane displaced from the object.

US 2013/0144137 and US 2014/0148658 present a system and method for use in monitoring one or more conditions of a subject's body. The system includes a control unit which includes an input port for receiving image data, a memory utility, and a processor utility. The image data is indicative of data measured by a pixel detector array and is in the form of a sequence of speckle patterns generated by a portion of the subject's body in response to illumination thereof by coherent light according to a certain sampling time pattern. The memory utility stores one or more predetermined models, the model comprising data indicative of a relation between one or more measurable parameters and one or more conditions of the subject's body. The processor utility is configured and operable for processing the image data to determine one or more corresponding body conditions; and generating output data indicative of the corresponding body conditions.

### GENERAL DESCRIPTION

There is a need in the art for a novel system and a technique enabling monitoring of mechanical and/or acoustic signals, for example such mechanical and/or acoustic vibrations associated with or originated from within a biological body. The present invention provides a system enabling remote sensing of vibrations (corresponding to acoustic and/or mechanical vibrations/signals) utilizing optical measurement that is non-invasive and provides accurate detection from relatively large distances as well as allows for storing digital data of the detected signals. Additionally, the technique of the present invention enables monitoring of micro-vibrations, or generally very small vibrations, which may be exposed or hidden behind a tissue layer.

The system is based on an optical measurement/collection system comprising a light source unit and a light collection unit. The optical measurement system generally utilizes coherent illumination of an inspection region (e.g. of a biological body such as a patient, human or animal) to be monitored, and collection of a secondary speckle pattern formed by scattering of light from the illuminated region. The speckle pattern is being collected by the collection unit, typically configured as a camera unit configured to be defocused with respect to the illuminated region. The collection unit is configured with an imaging lens arrangement and a detector array located for collecting image data associated with an intermediate plane between the inspection region and the imaging lens arrangement. This enables collection of image data pieces associated with secondary speckle patterns formed by interference of light components returning/scattering from the inspection region.

The present technique utilizes input data in the form of a sequence of image data pieces corresponding to so-collected speckle patterns for determining data about correlations between consecutive speckle patters indicative of variations in location or orientation of the inspection region. Such correlation function provides data on small variations in location and/or orientation of the illuminated region translated into variation in arrangement and/or location of the speckle pattern as collected by the collection unit. Such variations in location/orientation of the illuminated region may be indicative of vibrations of the inspection region or its surrounding and may be originating from acoustic signals or other mechanical vibrations arriving from vicinity of the region as well as of movement of the tissue or of neighboring tissues. Additionally, such variations in location/orientation of the tissue may be associated with mechanical (elastic or plastic) response of the tissue to an external stimulation.

The present technique utilizes the input data and the correlation function determined therefrom for determining data about acoustic signals collected from the inspection region, and generally the monitored body. To this end, data collected by the camera unit, generally by a corresponding detector array, is properly filtered and analyzed to differentiate between acoustic and other sources of movement, e.g. in accordance with frequency range and amplitude of vibrations. In this connection, it should be noted that camera sampling rate is preferably be sufficiently high for detection of acoustic signals, e.g. in accordance with Nyquist theorem and frequency range of the desired signals. The system of the present invention generally provides a control unit configured for receiving input data associated with time variations of secondary speckle pattern. The control unit generally comprises a processing utility, a memory utility as well as input and output ports providing connectivity to a network and/or user interface. The control unit is configured and operable for receiving input data from one or more camera units, filtering said input data to identify data associated with acoustic frequency pattern variation and generate corresponding output data indicative of detected acoustic signals.

Further, to enable the system for detecting and determining data about small internal or external movement of the sample, as well as for increasing signal to noise ratio in detection of acoustic signal, the present technique may also utilize adjustments or modulation to the coherent illumination of the light source directed at the inspection region. To this end, the technique of the present invention may also comprise a use of time modulated light source, configured to transmit coherent illumination pulsating at a predetermined frequency, selected in accordance with anticipated frequency of movement or of acoustic signals to be determined.

To this end the processing utility may generally include one or more software/hardware modules configured for applying selected frequency filtering to collected data and/or to operation of the light source unit as well as for determining correlations between collected speckle patterns and sample parameters. Thus, generally the processing utility comprises a correlation module and a filtering module, and may also include a frequency modulation module. The correlation module is configured and operable to receive a plurality of data pieces indicative of a sequence of speckle patterns collected over time and to determine spatial correlation between temporally consecutive speckle patterns. As indicated, variation in the speckle pattern provides an indication about movement, orientation variation or curvature variation of the illuminated region. The determined correlation function provides a relative indication of a measure of the variation in location, orientation and/or curvature of the region. To differentiate between changes associated with acoustic signals over mechanical movement or other movement, the filtering module is configured and operable to receive data stream indicative of the correlation function over certain period (typically in real time or minimal delay due to processing and determination of the correlation function) and to isolate acoustic frequencies within the pattern variation. The filtering module may operate to amplify acoustic frequencies while reduce amplitude of non-acoustic frequencies, as well as apply additional filtering to optimize detection of acoustic signals in accordance with operation directives. In some other embodiments, the technique or system may be operated for determining data about mechanical vibrations, which may be of lower frequency and/or similar frequency as acoustic signals (e.g. mechanical vibrations below 500 Hz (e.g. below 320 Hz or around 100 Hz). Additionally, the technique may utilizes external stimulation for determining data on stimulated vibrations with frequencies above 500 Hz which may be associated with tactile sensation of different texture as described in more details further below.

Additionally, the frequency modulation module, when used, is configured and operable to operate the light source unit to provide modulated pulsating coherent illuminations at a predetermined frequency. This is used to enable amplifying signal amplitude in a corresponding frequency (or an integer multiplication thereof) with respect to noise. It should be understood that the noise may typically be white noise, i.e. having all relevant frequencies. Thus, by amplifying a selected frequency, the light modulation may increase signal to noise ratio and assist in determining small variations (movement or acoustic data) in the sample.

According to some embodiments, the control unit may also include a signal selection module configured and operable for receiving data indicative of acoustic signals collected from the illuminated region and to apply selective filtering to thereby further isolate acoustic signals associated with one or more specific acoustic sources in accordance with operation objectives. For example, when the system is used to provide acoustic indication of cardiac activity, the signal selection module may be set to detect acoustic patterns associated with S1 and S2 characteristics corresponding to cardiac valves operation. Additionally or alternatively, the signal selection module may be configured to selectively identify and amplify signal data associated with additional known acoustic patterns such as breathing action and the activity of the lungs as well as muscle activity and bowl activity.

The technique of the invention may be used to determine various parameters associated with acoustic data, small movement data and mechanical response to external stimulation. As indicated, above, proper filtering may provide the system operation as a cardiac stethoscope, or stethoscope in general where the filtering and signal selection modules are configured to detect acoustic signals in corresponding frequencies and patterns. Additionally the system and technique of the invention may be used for monitoring of small movements and determine parameters of samples. For example, a paralyzed patient (e.g. patient with Amyotrophic Lateral Sclerosis (ALS) or other paralysis) may utilize the technique of the invention to detect small eye movements through a closed eyelid and thus enable the user to communicate with others effectively.

Additionally, the present technique may utilize an additional stimulating unit (e.g. acoustic or ultrasonic speaker) to determine tissue parameters such as bone density and elasticity or other parameters. In this connection, the illumination may be temporally modulated at a frequency corresponding to the stimulation frequency (e.g. similar or of integer multiplicity) to thereby amplify the desired signals over background noise that is may generally be white noise.

Additionally, as described above, the control unit may be connectable to various output units in accordance with type of signals selected for detections. In some embodiments, the system may be connectable to a tactile sensing unit configured for receiving data on detected vibrations from the control unit and for applying physical pressure variation providing a user with tactile sensation imitating the detected vibrations. This may provide insight about stiffness of the inspection region, or object associated therewith, and may assist a user (e.g. physician) in differentiating between different tissues, or tissue response.

Accordingly, the tactile sensing unit may for example be configured as a wearable sensing unit, e.g. glove, and comprise a plurality of actuators arranged in a predefined order to provide selected tactile sensing. The actuators are configured and operable for applying selected pressure in accordance with data received from the control unit to thereby imitate vibrations detected in a corresponding inspection region. Generally, the actuators may provide various types of tactile sensations in accordance with frequency and amplitude of the vibration detected from the inspection region.

In some configurations, the tactile sensing unit may also be configured to provide temperature variation sensation, e.g. comprise heating/cooling arrangement. The control unit may utilize remote temperature detection, e.g. using infra-red light detection, for determining temperature of the inspection region and provide corresponding information to the sensing unit.

Additionally or alternatively, the tactile sensing unit may also comprise a texture sensing region configured for providing texture sensing. Data about texture of the inspection region may be determined e.g. using external stimulation such as ultrasound stimulation, where texture variation data may be associated with vibration data (in response to the stimulation) having frequencies above 500 Hz. The tactile sensing unit may utilize one or more actuators for providing texture sensing to the user, and/or provide texture sensing using a dedicated texture sensing region enabling mechanically or electronically variated surface.

Thus, according to a broad aspect of the present invention there is provided a system for use in monitoring acoustic signals, the system comprising a control unit comprising: an input port for receiving a sequence of data pieces, each data piece being indicative of detected speckle pattern; and a processing module configured and operable for processing said sequence of data pieces to determine data about acoustic signals.

The system may further comprise, or be connectable to, at least one optical collection system, the at least one optical collection system comprises a light source configured to generate coherent illumination and direct said coherent illumination onto a region to be inspected, and a light collection unit comprising a lens unit and a detector array, the lens unit is configured to collect input light from an intermediate optical plane being in general direction along an axis between the inspected region and the detector array and to image said intermediate plane onto the detector array to thereby collect data about a secondary speckle pattern generated by scattering of the coherent illumination from the inspected region; the detector array is configured to detect said collected light to thereby generate image data of said secondary speckle pattern providing detected data pieces and to transmit the detected data pieces to the control unit.

According to some embodiments, the processing unit may comprise:

a correlation module configured and operable for receiving data about a sequence of input data pieces, each corresponding to a detected speckle pattern, and for processing the sequence of data pieces to determine correlation function between consecutive data pieces, said correlation function being indicative of variation in position of points within an inspected region;

a filtering module configured and operable for receiving data about the determined correlation function and to filter said data to identify position variation associated with acoustic signals originating for the inspected region to thereby generate data indicative of acoustic signals collected from said region.

The processing utility may further comprise a signal selection module, the signal selection module is configured and operable for receiving said data indicative of acoustic signals collected from said region, and for processing said data, selecting one or more acoustic signal patterns associated with an origin of the acoustic signals.

The system may be configured for use as a cardiac stethoscope, wherein the processing utility is configured and operable for identifying collected signals corresponding with acoustic indication of heart operation. It may also be configured to act as an accurate spirometer or a device listening to the breathing noises of the lungs to perform remote medical diagnostic related to lung activity. To this end the system may correlate the measure acoustic signals with one or more acoustic patterns associated with known selected biological activity, and/or with acoustic patterns determined during calibration.

According to some further embodiments, the system of the invention may be configured for use of nutrition by monitoring the chewing activity as part of total caloric consumption of a subject, wherein the processing utility is configured and operable for identifying collected signals corresponding with the chewing activity.

The system may also, according to some embodiments, be configured for use as a spirometer, wherein the processing utility is configured and operable for identifying collected signals corresponding with lungs activity.

Further, the system of the present invention may also be configured for use of sensing small eye movements, wherein the processing utility is configured and operable for identifying collected signals corresponding with eye movement as part of sleeping quality monitoring system.

According to one other broad aspect of the invention, there is provided a system for use in communication, the system comprising:
(a) an input port for receiving a sequence of data pieces, each data piece being indicative of detected speckle pattern; and a processing module configured and operable for processing said sequence of data pieces to determine data about small movements to thereby interpret said small movements to communication massages.

The system may further comprise at least one optical collection system, the at least one optical collection system comprises a light source configured to generate coherent illumination at a selected frequency and direct said coherent illumination onto a region to be inspected, and a light collection unit comprising a lens unit and a detector array, the lens unit is configured to collect input light from an intermediate optical plane being in general direction along an axis between the inspected region and the detector array and to image said intermediate plane onto the detector array to thereby collect data about a secondary speckle pattern generated by scattering of the coherent illumination from the inspected region; the detector array is configured to detect said collected light to thereby generate image data of said secondary speckle pattern providing detected data pieces and to transmit the detected data pieces to the control unit. The region to be inspected may be a user's closed eyelid.

According to some embodiments, the processing unit may comprise:
a correlation module configured and operable for receiving data about a sequence of input data pieces, each corresponding to a detected speckle pattern, and for processing the sequence of data pieces to determine correlation function between consecutive data pieces, said correlation function being indicative of variation in position of points within an inspected region;
a frequency selection module configured and operable for selecting an operation frequency for modulation of the light source unit and sampling rate of the camera unit in accordance with data about anticipated frequency of movement to be detected.

According to some embodiments, the processing unit may further comprise a filtering module configured and operable for receiving data about the determined correlation function and to filter said data to identify position variation associated with movement signals originating for the inspected region to thereby generate data indicative of movement patterns signals collected from said region.

According to some embodiments, the processing utility may further comprise a signal selection module, the signal selection module is configured and operable for receiving said data indicative of signals collected from said region, and for processing said data, selecting one or more signal patterns associated with an origin of the movement signals.

According to some embodiments, the system may be configured for use of sensing small eye movements, wherein the processing utility is configured and operable for identifying collected signals corresponding with eye movement as part of sleeping quality monitoring system.

According to yet another broad aspect of the invention, there is provided a system for inspection of bone density, the system comprising:

a light source unit configured to provide time modulated coherent illumination to be directed at a bone to be inspected;

a camera unit configured to collect light returning from said bone, the camera comprises a detector array and an optical arrangement configured to provide defocus imaging of said light returning from the sample, to thereby provide image data corresponding to a secondary speckle pattern of scattered light;

a stimulating unit configured to provide acoustic stimulation at a selected frequency to said bone; and

a control unit configured and operable to determine a selected operation frequency to the light source unit, camera unit and stimulating unit, and to receive a plurality of image data pieces from the camera unit, each being indicative of a secondary speckle pattern, and to determine correlation between consecutive speckle patterns to thereby determine the bone's elastic or mechanical response to stimulation from the stimulating unit.

The control unit may be further configured to determine state of bone density or state of osteoporosis of said bone.

According to yet another broad aspect the present invention provides a system comprising: input port for receiving input data comprising a sequence of image data pieces indicative of speckle patterns detected in light returning from an inspection region, at least one processing utility configured for processing and analyzing the input data, the at least one processing utility comprises: a correlation module configured for processing the sequence of image data piece and determining correlation function indicative of spatial correlations between speckle patterns in consecutive image data pieces, said correlation function being indicative of variation in position of points within an inspected region; a filtering module configured and operable for receiving and processing data on the determined correlation function for extracting data indicative on vibration having selected frequency range for determining data indicative of at least one of acoustic signals, movement and tactile vibrations of said inspection region.

In some embodiments, the system may further comprise at least one optical collection system, the at least one optical collection system comprises a light source configured to generate coherent illumination at a selected frequency and direct said coherent illumination onto a selected inspection region, and a light collection unit comprising an imaging lens arrangement and a detector array, the an imaging lens arrangement is configured for collecting input light returning from said inspection region to thereby generate on the detector array an image corresponding to an intermediate plane located between the inspection region and said imaging lens arrangement, the detector array is configured to detect said collected light to thereby generate image data associated with secondary speckle patterns generated by scattering of the coherent illumination from the inspected region and for generating and transmitting corresponding image data pieces providing detected data pieces for processing, the light collection unit is thereby configured for collecting image data pieces indicative of secondary speckle patterns.

The optical collection system may be configured for use with an inspection region being a portion of a human body. The portion of a human body may be at least one of: closed eyelid, jaw, chest, hand, neck, forehead and forehead-temple.

According to some embodiments, the processing utility may further comprise a signal selection module, the signal selection module is configured and operable for receiving said data indicative of one or more selected movement types collected from said region, and for processing said data, selecting one or more movement patterns associated with a desired movement origin. The movement/vibration pattern may be associated with acoustic signals, said desired movement origin being a desired origin of acoustic signals to be detected.

The processing utility may further comprise a frequency selection module configured and operable for selecting an operation frequency for modulation of the light source unit and sampling rate of the camera unit in accordance with data about anticipated frequency range of movement to be detected.

The filtering module may be configured and operable for receiving data about the determined correlation function and for filtering said data to identify position variation associated with movement signals originating for the inspection region to thereby generate data indicative of movement patterns signals collected from said region.

According to some embodiments the system may be configured for use of sensing small eye movements, wherein the processing utility is configured and operable for identifying collected signals corresponding with eye movement as part of sleeping quality monitoring system.

According to some embodiments the system may be configured for use as a cardiac stethoscope, said processing utility is configured and operable for identifying collected signals corresponding with acoustic indication of heart operation.

According to some embodiments the system may be configured for use of nutrition by monitoring the chewing activity as part of total caloric consumption of a subject, said processing utility is configured and operable for identifying collected signals corresponding with the chewing activity.

According to some embodiments the system may be configured for use as a spirometer, said processing utility is configured and operable for identifying collected signals corresponding with lungs activity.

According to some embodiments the system may be configured for use of sensing small eye movements, said processing utility is configured and operable for identifying collected signals corresponding with eye movement as part of sleeping quality monitoring system.

According to some embodiments, the system may further comprise an output port connectable to a tactile sensing unit, said tactile sensing unit comprising a plurality of actuation element configured for operating in response to data indicative of detected vibrations from said inspection region, to thereby provide tactile sensation to a user in accordance with said collected vibration data.

The tactile sensing unit may be configured as a wearable unit and configured for providing tactile sensing to a user in accordance with vibrations detected at said inspection region. The tactile sensing unit may be configured for providing vibration sensation of at least one of low frequency movement, medium frequency vibrations.

Generally tactile sensing unit may be configured for providing vibration sensation associated with vibration frequency of 500 Hz and higher enabling texture-like sensation. To this end the tactile sensing unit may utilize high-frequency actuators and/or comprise a texture sensing element configured for mechanically or electronically vary texture of a selected surface thereof, to thereby provide corresponding texture sensing to the user.

According to some embodiments, the tactile sensing unit may comprise high frequency actuation element configured for providing tactile sensing associated with texture of the inspection region utilizing data indicative of high frequency vibration in response to external stimulation of the inspection region. Additionally or alternatively, the tactile sensing unit may comprise at least one texture sensing element configured for mechanically or electronically vary texture of a selected surface thereof in response to input data indicative of high frequency vibrations of the inspection region.

According to some embodiments, the tactile sensing unit may comprise a plurality of at least two actuation regions comprising at least two of low-frequency pressure sensing, mid-frequency vibration sensing, texture variation sensing and temperature sensing.

According to some embodiments, the system may further comprise a stimulation unit configured for providing selected external stimulation onto said inspection region, said external stimulation being associated with acoustic signals of selected frequency range. The processing utility may further comprise a stimulation selection module configured and operable determining said selected frequency of the external stimulation, and an elastometry/elastography module configured and operable for receiving data about correlation between consecutive speckle patterns filtered in accordance with said selected frequency for determining data about material density of said inspection region.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates schematically a system for inspection of a region according to some embodiments of the invention;

FIG. 2 illustrates a control unit for use in detection of acoustic signals according to some embodiments of the invention;

FIGS. 3A and 3B show spectrogram representation (FIG. 3A) of detected acoustic signals and detected acoustic signal (FIG. 3B) associated with cardiac activity of a patient;

FIG. 4 shows acoustic signal associated with jaw joints movement collected using the technique of the present invention;

FIG. 5 shows acoustic signals associated with eye movement that may be related to sleep quality monitoring and which is being collected using the technique of the present invention;

FIGS. 6A and 6B exemplify a use of the technique of the present invention for effective communication of immobile, e.g. paralyzed, patients using minor eye movements;

FIG. 7 schematically illustrates a tactile sensing unit configured for providing tactile data to a user according to some embodiments of the invention; and

FIG. 8 shows a schematic flow diagram describing the technique of the present invention.

### DETAILED DESCRIPTION OF EMBODIMENTS

Reference is made to FIG. 1 schematically illustrating a system 1000 for use in remote collection of signals, associated with vibrations or movement (e.g. acoustic), from a target. The system generally includes a coherent light source 200 configured to be directed at a region R of the sample to be inspected and a camera unit 300 configured to collect light reflected/scattered from the inspected region R and may include a stimulating unit 400 configured to transmit certain stimulation onto the sample. The system 1000 may also include an output module 850 (user interface module) configured for providing output of a selected form to a user. The camera unit 300 is configured to be defocused with respect to the region R to thereby provide imaging of an intermediate plane P located along optical path between the region R and the camera unit 300. The camera unit generally includes a detector array (not specifically shown) configured to collect image data associated with a secondary speckle pattern collected from the intermediate plane P as describe in more details in U.S. Pat. No. 8,638,991 and in US patent publications US 2013/0144137 and US 2014/0148658 incorporated herein by reference.

The light source 200 and the camera unit 300, and the stimulating unit 400 (when used), are connectable to a control unit 500 configured and operable for operating the light source 200 and camera unit 300 and stimulating unit 400 and for receiving data stream from the camera unit 300, indicative of sequence of image data pieces of collected speckle patterns. The control unit 500 generally includes a processing module configured for processing and analyzing the collected data stream to determine data about acoustic signals formed at the inspected region R.

In this connection, it should be noted that the region R may generally be a region of any sample. More specifically, the technique and system of the invention may advantageously be used to provide acoustic sensing of living organisms and in particular of human patients. Thus, the system of the present invention may be directed at a patient or a body part of a patient (e.g. closed eyelid, chest region, neck or any other region on the patient's body) or to any other biologic tissue, and provide remote and invasive detection of material properties and/or various internal processes by detecting mechanical and acoustic signals in a similar manner that a physician used a stethoscope to detect biomechanical operation such as cardiac activity, joint movement, bowl activity etc. In this connection, the commonly used stethoscope, although providing a simple and effective diagnosis tool, suffers from disadvantages such as the lack of documentation of the detected signals, personal interpretation of the physician and the requirement for direct contact with the inspected region on the patient. The technique of the invention provides for remote sensing of acoustic activity, enables storing of the collected data for comparison and monitoring and may thus be used for continuous monitoring as well as on-the-fly monitoring of patients.

Additionally, the present technique may be used in surgical operation by inspecting a selected tissue and determining data on vibration (or response to external stimulation) thereof, and provide the detected data in the form of tactile stimulation to a user utilizing a tactile sensing unit connectable as output module 850 to the system. Generally, the output module 850 may be associated with a user interface unit (e.g. screen) or communication network connection. In some embodiments, the output module may include a tactile sensing unit configured to provide tactile stimulation indicative of vibrations detected at the inspection region R.

Further according to some embodiments, the system and technique of the invention may be used to enable remote communication system for patients. For example, a paralyzed patient may utilize the system as described herein for detecting small movements of his eyes behind closed eyelids to communicate with his surroundings. To this end, the system may be configured to direct the coherent light source 200 to illuminate a region on or next to the user's closed eyelid, to enable detection of movements of the user's eyes behind the closed eyelid. The system may be configured to interpret eyes movement at simple YES and NO answers or determine more complex thoughts based on the user's training and predefined algorithms.

In order to enable the system to determine such small movements, as well as additional parameters and properties, the present invention may also utilize temporal modulation of the sample illumination by the light source unit 200. More specifically, the light source unit may be configured to be operated by the control unit in a pulsating fashion at a frequency selected in accordance with preexisting data about the properties to be determined, e.g. similar frequency, corresponding Nyquist frequency or an integer multiplicity of the signal frequency. Respectively, the sampling rate of the camera unit 300 may be similarly modulated to provide corresponding sapling rate. This enables amplification of signals of respective frequencies with respect to signals or noise of other frequencies. As the general noise may be white noise, the resulting effect may be increased Signal to noise ratio (SNR).

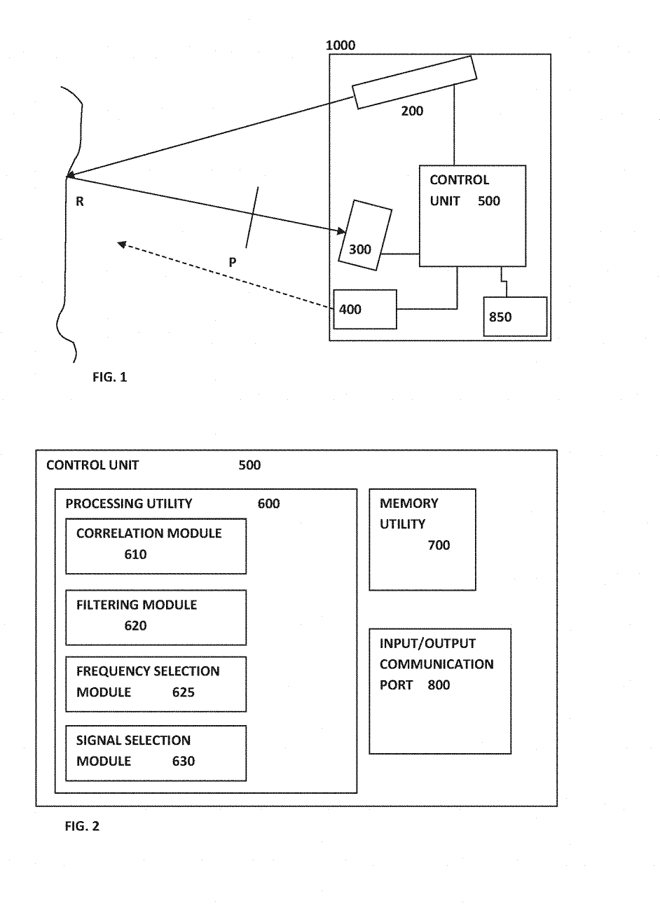

It should also be noted that the technique of the present invention also provides a system configured as a control unit 500 and connectable to one or more sensing units where each of the sensing units includes a coherent light source 200, a camera unit 300 and in some configurations one or more stimulating unit 400, and is configured to be directed at a region to be inspected, for a single or a plurality of patients. The control system is exemplified in FIG. 2 and comprising a processing utility 600 and input/output communication ports 800 for receiving input data and for providing output data through network communications and/or user interface, e.g. utilizing the output module 850 in FIG. 1.

The control system 500 illustrated in FIG. 2 typically includes a processing utility including software or hardware modules for processing and analyzing the input data to provide output data indicative of acoustic signals detected from the one or more inspected regions. More specifically, the processing utility 600 includes a correlation module 610 and a filtering module 620. According to some embodiments the processing utility may also include a frequency selection module 625 and/or a signal selection module 630.

The correlation module 610 is configured and operable for receiving input data in the form of a sequence of data pieces, each indicative of an image data of a collected speckle pattern. The sequence is collected at a certain temporal sampling rate to provide indication of temporal variations at the inspected region. The correlation module 610 processes the input sequence of data pieces to determine spatial correlation between consecutive speckle patterns thereby determining a measure of variation in consecutive collected patterns. Such variation between arrangements of the speckle patterns is typically indicative of variations within the reflecting/scattering surface of the inspected region R associated with movement, change in orientation or in curvature of the surface of the region. The determined correlation functions between a plurality of consecutive pairs of collected data pieces (associated with a plurality of speckle patterns) is indicative of variations in the inspected region over time.

The correlation module 610 typically transmits data about correlation function between consecutive input data pieces (speckle patterns) to the filtering module 620. The filtering module is configured and operable for receiving the correlation function data and for processing and analyzing it to identify data indicative of acoustic signals that may be generated at, or around, the inspected region. Similarly to a stethoscope, capable of providing acoustic data associated with internal activity and collected from the surface of the body, the technique of the present invention provides data about (typically) internal acoustic signals detected from the surface of the inspected region by variations in location, curvature and orientation of the region.

To this end, the filtering module 620 is configured to apply filtering to the determined correlation function to select correlation variation occurring at frequencies associated with selected signals such as acoustic frequencies, vibrations, texture etc. Typically, the relevant acoustic frequencies may be between 1 Hz and 20 KHz, relevant frequencies associated with tactile sensation may be below 250 Hz, or below 320 Hz for vibration/motion or higher than 500 Hz that are typically not directly sensed by tactile sensing and represent texture variation, in some configuration the filtering module 620 is configured to select a band within or outside of the acoustic frequency range such as correlation variations occurring at frequencies between 0.5 Hz and 1 KHz, or between 20 Hz and 500 Hz, or around 400 Hz etc. Generally, the filtering module is configured to apply low-pass filtering corresponding to acoustic signals detected through a biological tissue. It should also be noted that the maximal variation frequency detectable is limited by the sampling rate of the camera unit 300 in accordance with Nyquist theorem.

The control system 500 may provide output data indicative of the detected acoustic signals in the form of graphical data on a display unit and/or in the form of acoustic output data enabling physicians to listen in a similar manner as they might be used to. Additionally, the control system 500 may store and transmit the collected acoustic data for storing and further processing as the case may be.

As also shown in FIG. 2, the control system 500 may also include a frequency selection module 625 and a signal selection module 630. The frequency selection module is configured and operable for receiving data indicative of selected operational/sampling frequency (e.g. from an operator or pre-stored data stored in the memory unit 700) and to operate the light source unit 200, the camera unit 300 and in some configurations, the stimulating unit 400, at a frequency corresponding to the selected operational/sampling frequency. More specifically, the frequency selection module 625 may operate the light source unit 200 to provide pulsating illumination at a predetermined frequency, e.g. selected frequency between 50 Hz and 500 Hz, or below 50 Hz, or above 500 Hz, etc. The frequency selection module 625 also operates the camera unit 300 at a substantially similar frequency to allow proper detection of variations in speckle patterns of light scattered from the inspected region. The use of selected stimulating frequency, in combination with corresponding sampling frequency (of the light source as well as the camera unit) provides effective “analog” amplification of signal having similar or corresponding frequencies while white noise is left low. This provides for increased SNR and sensitivity of the system for detection and inspection of samples/tissues/users. Such stimulation modulation may for example be used to finely determined bone density and elastic characteristics of it to determine state of osteoporosis or other bone related diseases.

The signal selection module 630 may be configured and operable for receiving data indicative of detected acoustic signals and for processing the data to identify and determine acoustic patterns corresponding to one or more parameters to be identified. More specifically, the signal selection module 630 may be preconfigured to identify acoustic signals associated with cardiac activity such as S1 and S2 signals and to amplify corresponding frequencies to provide enhanced output. Thus, the signal selection module may apply signal processing and matching to detect and enhance signals associated with predefined known acoustic patterns such as S1 and S2 components of heart beats, muscle peristalsis (e.g. gastrointestinal activity), jaw movement and air flow into and out of the lungs (e.g. providing a non-contact optical spirometer).

It should be noted that the selection of acoustic patterns may or may not be associates with the exact location of the inspected region R. For example, while monitoring cardiac activity the region R may preferably be at the chest, it may also be from other portions of the body as the blood flow transmits acoustic data and as the signal selection module 630 may operate to enhance acoustic signals associated with the desired pattern. However, since muscle activity may be originated from effectively any muscle in the patient's body, when searching for bowl activity or other muscle activity, it is preferable to select the inspected region R to be in close proximity with the selected activity origin.

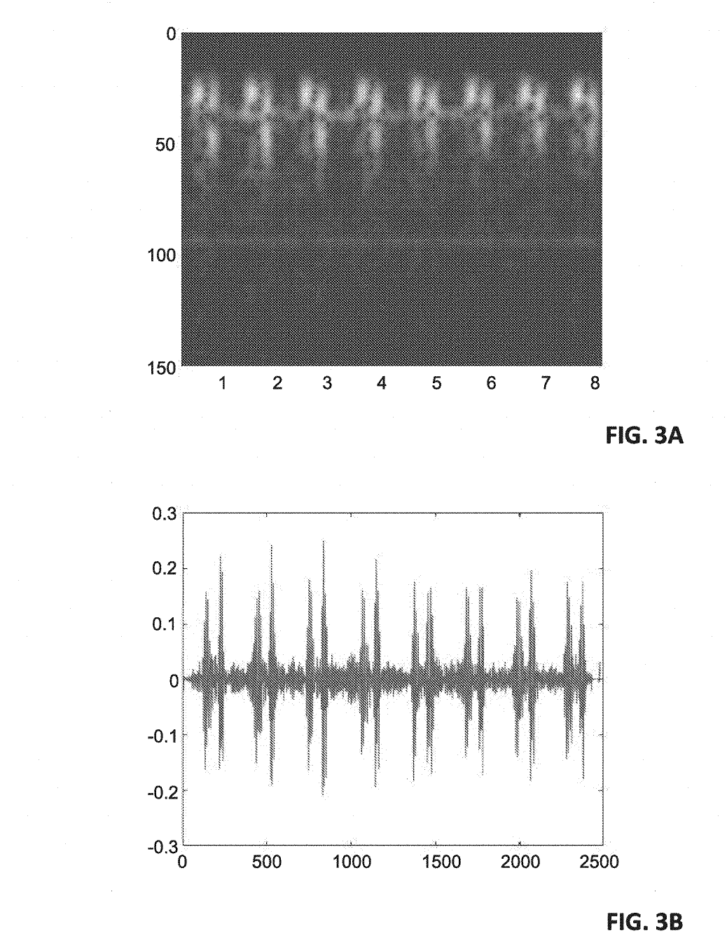

To exemplify operation of the above described technique, FIGS. 3A and 3B show processed measured data associated with cardiac activity as remotely measured (at a distance of about 1.5 meters from the subject) by the present technique. FIG. 3A shows a spectrogram of collected signals showing the frequency components on the vertical axis, time on the horizontal axis and where the amplitude of each frequency component at certain time is shown by color of the spectrogram (white is greater amplitude and dark is lower amplitude). As seen in the spectrogram of FIG. 3A, there is a sequence of acoustic pulses arriving in pairs, in a similar manner to S1 and S2 components detected in cardiac activity. FIG. 3B shows a measured temporal signal at the corresponding frequency range around 400 Hz, clearly showing the components of cardiac activity. Such acoustic sensing may also provide indication of cardiac malfunction that may be indicated by elongated acoustic signals, murmur signals or other variations as generally known in the medical literature.

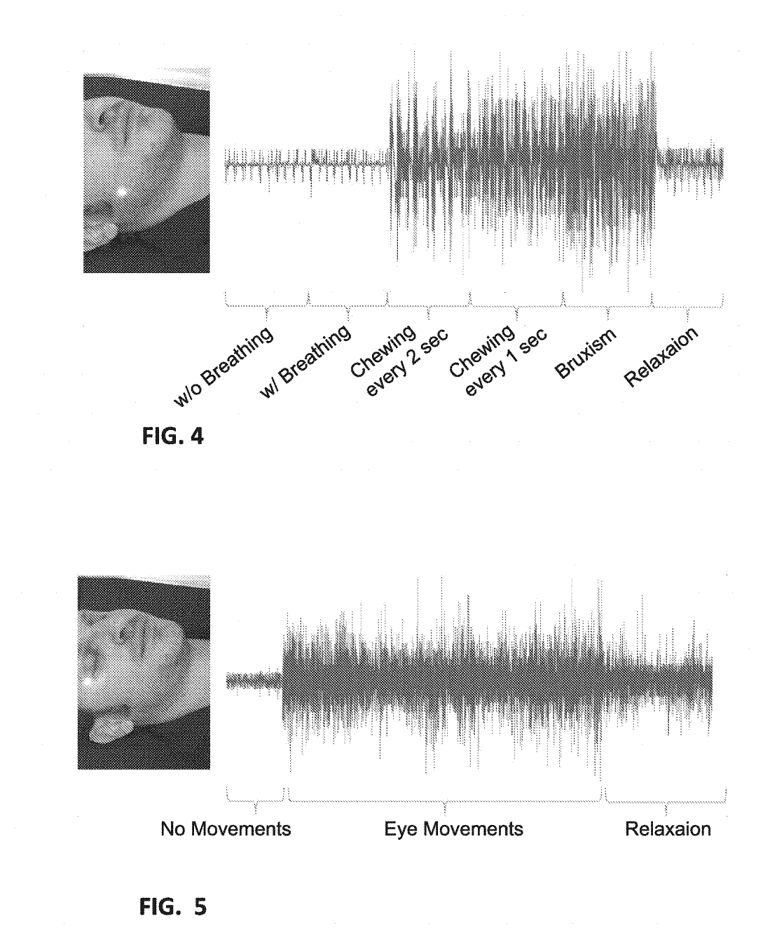

As also indicated above, the present technique may provide acoustic data associated with additional activities in a patient. Reference is made to FIG. 4 and FIG. 5 showing acoustic signals detected from a patient's jaw as marked in illuminated spot on the patient in FIG. 4 and patient's eye as marked by corresponding illuminated spot in FIG. 5. The figures show detected acoustic activity while the patient is asked to perform certain tasks for calibration. In FIG. 4, the patient was asked to hold breathing (w/o breathing), breath regularly (w/ breathing), slowly move his law for chewing every 2 seconds and every second (Chewing every 2 sec and Chewing every 1 sec), grind his teeth (Bruxism) and relax his jaw (Relaxation). As can be seen, the detected acoustic signals vary between movement types and can be used to determine patient's jaw activity. It should be noted that the spot of light visible in the FIGS. 4 and 5 indicate the region being illuminated by the laser and which correspond to the tissue from which the remote sensing procedure is being performed.

Similarly, the system was directed at the patient's eye as shown in FIG. 5. The detected acoustic signals were collected with the patient being asked not to move his eye (No Movement), move his eyes rapidly (Eye Movement) and relax his eyes (Relaxation). The detected acoustic signals provide indication of the eye's movement from a distance without the need to touch the patient. This parameter may for example be important for properly monitoring the sleep quality.

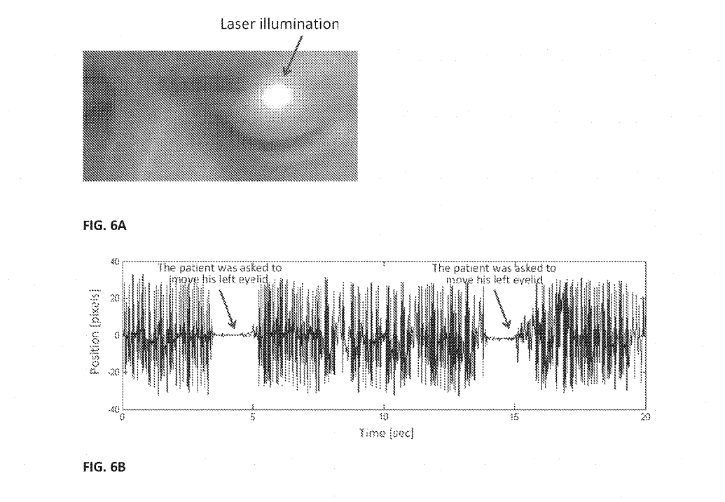

Reference is made to FIGS. 6A and 6B illustrating a use of the above described technique for communication with paralyzed patient, e.g. having final stage ALS. FIG. 6A shows direction of the coherent illumination on the user's closed eyelid, to determine movements of the eye behind the closed eyelid, and FIG. 6B shows experimental data about eye movement detection by the system of the invention. In the measurement shown here, the system is configured to illuminate the inspection region (eyelid of the user) at a modulated pulsating frequency of about 400 Hz, where the camera unit is operated at a corresponding sampling rate of 400 fps (frames per second). The correlation between consecutive speckle patterns provides data indicative of position or orientation of the user's eyelid. In the position vs. time graph shown in FIG. 6B the position data is filtered to illustrate high frequency movements of the eye, associated with involuntary eye movements at rest. As shown, when at rest, the user's eyes are flickering around while when asked to move his eyelid, the flickering stops and low frequency movement can be shown. The above described technique may utilize various other frequency filters and user instructions to provide higher level communication using eye and eyelid movement, thus enabling simple communication to ALS patients and otherwise paralyzed users.

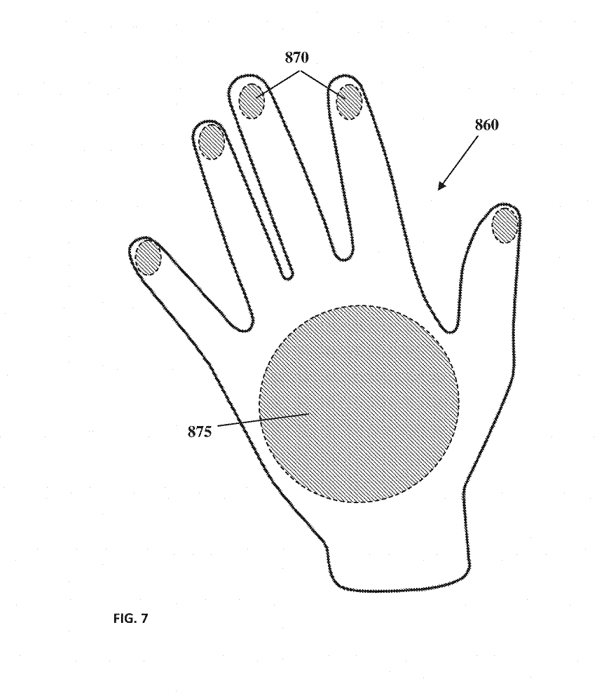

Reference is made to FIG. 7 illustrating a glove shaped tactile sensing unit 860 configured for providing tactile sensation output according to some embodiments of the invention. The tactile sensing unit 860 is generally configured with a plurality of actuation elements 870 (actuators) such as piezo-electric elements or motorized actuators. The tactile sensing unit may also include texture and/or temperature sensing element 875 provided heating/cooling sensing and/or providing sensing of texture variation in accordance with collected data associated with high frequency vibrations collected from the inspection region. As indicated above, the system of the present invention is configured for determining vibration data in one or more selected frequencies utilizing optical inspection and collection of speckle patterns from an inspection region.

In this example the tactile sensing unit 860 is exemplified as a glove-like unit and includes actuators 870 located at regions associated with increased sensing. In some other configurations, the tactile sensing unit may be configured for applying pressure data on a user's forehead (in a hat-like or spectacles configuration) or any other body part of the user. This configuration enables a user for remotely feeling vibration (internal or in response to external stimulation as described above) and/or certain textures of a tissue, while not physically being in direct contact. For example, the system may utilize a stimulation unit (400 in FIG. 1) configured for providing ultrasound stimulation to one or more selected region of a patient, and collect data about vibrational response of a selected tissue (inspection region). Output data about detected vibration response may be transmitted to the tactile sensing unit 860 operating the actuators 870, and in some embodiments the texture sensing element 875, thereof to provide a user (e.g. a surgeon) with sensory data on the tissue being inspected. Such non-contact elastography sensing may provide insight regarding the stiffness of the inspected objects/tissues without requiring a physician to actually touch the region, thus enabling increased accuracy in remote/robotic surgery.

Generally, the actuators 870 may be configured for operating in a wide range of frequencies. Alternatively, the tactile sensing unit may be configured with an array of actuators having different frequency ranges enabling transmission of sensory data in various frequencies. Typically, human sense of touch is sensitive to vibrations between 60 Hz and 500 Hz, and especially between 100 Hz and 320 Hz. It should however be noted that the present technique may also utilize transmission of tactile sensory data at frequencies higher than 500 Hz, providing a user data about texture of the inspection region. Such texture sensing may be provided by activating an array of actuators 870 at high frequency, and/or using a texture sensing element 875 configured for varying a selected surface to provide feeling of texture variation.

Generally, the technique may utilize frequency variation and/or multiplexing of external stimulations for determining different types of textures of possible inspection regions. This enables a user (e.g. surgeon) to differentiate between different types of tissues based on changes in texture as determined by corresponding elastography data collected in response to the external stimulation.

Further, the tactile sensing unit 860 may be configured with various sensing regions configured for providing tactile stimulation associated with selected sensing parameters. For example, the tactile sensing unit 860 may include low-frequency pressure sensing, mid-frequency vibration sensing, texture variation sensing and temperature sensing, wherein each of the different actuation regions in configured with suitable actuation element having the corresponding frequency range and structure for providing reliable sensing feeling to the user.

In some embodiments, the system may also include an infrared thermometer, configured for remotely measuring temperature of the inspection region. Additionally, the tactile sensing unit 860 may include cooling/heating elements configured for providing sensory data on temperature to the user, providing feel of the tissue as well as its temperature, or temperature variations.

Generally, these configurations may be used for medical uses, e.g. for remote/robotic surgery. Additionally, such configuration may also be used for non-contact inspection of various other elements where touch would be the state of the art inspection technique such as quality of food products such as vegetables or fruits etc., as well as for entertainment uses such as gaming or remote interaction between users.

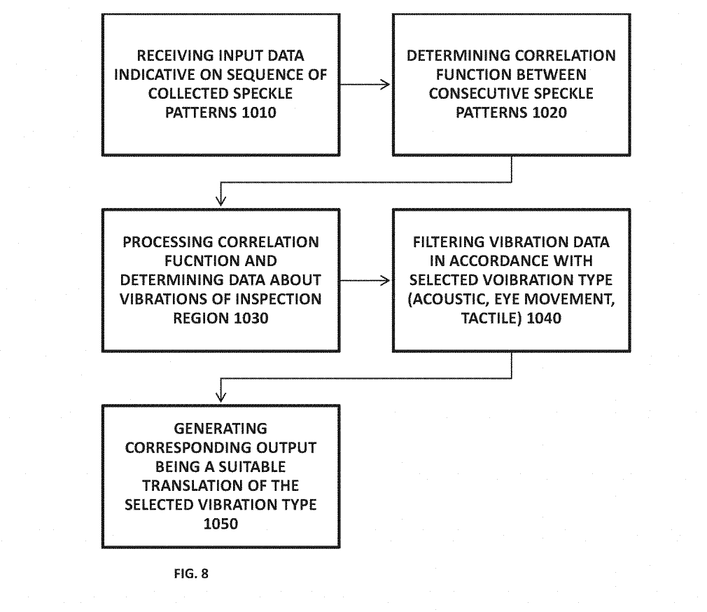

Reference is made to FIG. 8 showing a flow chart illustrating the technique of the present invention. As shown, input data indicative of a sequence of image data pieces associated with speckle patterns collection from an inspection region is provided 1010. The input data is processed by determining correlation function between consecutively collected speckle patterns 1020, the correlation function is indicative of vibrations and movement data of the inspection region 1030. The vibration data may generally include noise and data about vibrations other than one or more desired signals (acoustic and/or mechanical vibrations) and the technique further includes filtering the vibration data 1040 based on frequency range, and possibly amplitude (strength) of the vibrations for differentiating between acoustic, movement types and noise data. Data about the desired signal is extracted by the filtering and being output 1050 via a suitable output unit to a user. Such output unit may be speaker system, screen (showing graphical data) as well as tactile unit.

Such monitoring technique may be used for monitoring heart activity, breathing activity, sleep and nutrition monitoring as well as routine checkup without the need to approach the patient and/or touch him. Further, the present technique may be used for determining tissue parameters and/or provide movement-based communication using eye or eyelid movement or any other micro-movement of a user. It should be noted that the wavelength of the coherent illumination may typically be any wavelength between the optical spectrum and near IR. This is to allow simple detection and enable the use of optical instruments such as lenses and detector arrays.

1. A system comprising:
input port for receiving input data comprising a sequence of image data pieces indicative of speckle patterns detected in light returning from an inspection region,
at least one processing utility configured for processing and analyzing the input data, the at least one processing utility comprises:
i) a correlation module configured for processing the sequence of image data piece and determining correlation function indicative of spatial correlations between speckle patterns in consecutive image data pieces, said correlation function being indicative of variation in position of points within an inspected region;
ii) a filtering module configured and operable for receiving and processing data on the determined correlation function for extracting data indicative on vibration having selected frequency range for determining data indicative of at least one of acoustic signals, movement and tactile vibrations of said inspection region.

2. The system of claim 1, further comprising at least one optical collection system, the at least one optical collection system comprises a light source configured to generate coherent illumination at a selected frequency and direct said coherent illumination onto a selected inspection region, and a light collection unit comprising an imaging lens arrangement and a detector array, the an imaging lens arrangement is configured for collecting input light returning from said inspection region to thereby generate on the detector array an image corresponding to an intermediate plane located between the inspection region and said imaging lens arrangement, the detector array is configured to detect said collected light to thereby generate image data associated with secondary speckle patterns generated by scattering of the coherent illumination from the inspected region and for generating and transmitting corresponding image data pieces providing detected data pieces for processing, the light collection unit is thereby configured for collecting image data pieces indicative of secondary speckle patterns.

3. The system of claim 2, where said optical collection system is configured for use with an inspection region being a portion of a human body.

4. The system of claim 3, wherein said portion of a human body being at least one of: closed eyelid, jaw, chest, hand, neck, forehead, forehead-temple.

5. The system of claim 1, wherein said processing utility further comprises a signal selection module, the signal selection module is configured and operable for receiving said data indicative of one or more selected movement types collected from said region, and for processing said data, selecting one or more movement patterns associated with a desired movement origin.

6. The system of claim 5, wherein said movement pattern being associated with acoustic signals, said desired movement origin being a desired origin of acoustic signals to be detected.

7. The system of claim 1, wherein said processing utility further comprises a frequency selection module configured and operable for selecting an operation frequency for modulation of the light source unit and sampling rate of the camera unit in accordance with data about anticipated frequency range of movement to be detected.

8. The system of claim 1, wherein the filtering module is configured and operable for receiving data about the determined correlation function and for filtering said data to identify position variation associated with movement signals originating for the inspection region to thereby generate data indicative of movement patterns signals collected from said region.

9. The system of claim 1, configured for use of sensing small eye movements, wherein the processing utility is configured and operable for identifying collected signals corresponding with eye movement as part of sleeping quality monitoring system.

10. The system of claim 1, configured for use as a cardiac stethoscope, said processing utility is configured and operable for identifying collected signals corresponding with acoustic indication of heart operation.

11. The system of claim 1, configured for use of nutrition by monitoring the chewing activity as part of total caloric consumption of a subject, said processing utility is configured and operable for identifying collected signals corresponding with the chewing activity.

12. The system of claim 1, configured for use as a spirometer, said processing utility is configured and operable for identifying collected signals corresponding with lungs activity.

13. The system of claim 1, configured for use of sensing small eye movements, said processing utility is configured and operable for identifying collected signals corresponding with eye movement as part of sleeping quality monitoring system.

14. The system of claim 1, further comprising an output port connectable to a tactile sensing unit, said tactile sensing unit comprising a plurality of actuation element configured for operating in response to data indicative of detected vibrations from said inspection region, to thereby provide tactile sensation to a user in accordance with said collected vibration data.

15. The system of claim 14, wherein said tactile sensing unit being configured as a wearable unit and configured for providing tactile sensing to a user in accordance with vibrations detected at said inspection region.

16. The system of claim 14, wherein said tactile sensing unit is configured for providing vibration sensation of at least one of low frequency movement and medium frequency vibrations.

17. The system of claim 14, wherein said tactile sensing unit is configured for providing vibration sensation associated with vibration frequency of 500 Hz and higher enabling texture-like sensation.

18. The system of claim 17, wherein said tactile sensing unit comprises high frequency actuation element configured for providing tactile sensing associated with texture of the inspection region utilizing data indicative of high frequency vibration in response to external stimulation of the inspection region.

19. The system of claim 17, wherein said tactile sensing unit comprises at least one texture sensing element configured for mechanically or electronically vary texture of a selected surface thereof in response to input data indicative of high frequency vibrations of the inspection region.

20. The system of claim 14, wherein said tactile sensing unit comprises a plurality of at least two actuation regions comprising at least two of low-frequency pressure sensing, mid-frequency vibration sensing, texture variation sensing and temperature sensing.

21. The system of claim 1, further comprising a stimulation unit configured for providing selected external stimulation onto said inspection region, said external stimulation being associated with acoustic signals of selected frequency range.

22. The system of claim 21, wherein said processing utility further comprises a stimulation selection module configured and operable determining said selected frequency of the external stimulation, and an elastometry module configured and operable for receiving data about correlation between consecutive speckle patterns filtered in accordance with said selected frequency for determining data about material density of said inspection region.

23. A system for inspection of bone density, the system comprising:
a light source unit configured to provide time modulated coherent illumination to be directed at a bone to be inspected;
a camera unit configured to collect light returning from said bone, the camera comprises a detector array and an optical arrangement configured to provide defocus imaging of said light returning from the sample, to thereby provide image data corresponding to a secondary speckle pattern of scattered light;
a stimulating unit configured to provide acoustic stimulation at a selected frequency to said bone; and
a control unit configured and operable to determine a selected operation frequency to the light source unit, camera unit and stimulating unit, and to receive a plurality of image data pieces from the camera unit, each being indicative of a secondary speckle pattern, and to determine correlation between consecutive speckle patterns to thereby determine the bone's elastic or mechanical response to stimulation from the stimulating unit.

24. The system of claim 23, wherein said control unit being further configured to determine state of bone density or state of osteoporosis of said bone.
